(19) United States
(12) Patent Application Publication
(10) Pub. No.: US 2008/0221549 A1
(43) Pub. Date: Sep. 11, 2008

(54) RAPID INJECTION DEVICE

(76) Inventor: Nahum COHEN, Karme Yosef (IL)

Correspondence Address: DANIEL J SWIRSKY, 55 REUVEN ST., BEIT SHEMESH, 99544, IL

(21) Appl. No.: 12/042,501

(22) Filed: Mar. 5, 2008

Related U.S. Application Data
(60) Provisional application No. 60/893,159, filed on Mar. 6, 2007.

(51) Int. Cl. A61M 31/00; A61M 5/178

(52) U.S. Cl. 604/506; 604/187

(57) ABSTRACT

A rapid injection device including a hand-held injection unit having multiple needles to inject at least one subject, and an injection manager to control at least the injection unit according to injection parameters, such as for performing rapid injections by receiving a mode of operation, receiving a dosage amount, receiving a needle extension length, extending a needle the length of the needle extension length out a needle storage unit, and pumping the dosage amount of fluid through the needle.

### FIELD OF THE INVENTION

The present invention relates to injection devices generally.

### BACKGROUND OF THE INVENTION

When injections are administered to a large number of animals, the same needle is often repeatedly used. This is due primarily to the need to carry out mass injections quickly and efficiently, whereas employing multiple needles is time-consuming, cumbersome, and potentially unsafe due to the burden their disposal places upon those giving the injections. However, reusing the same needle is unsanitary and may lead to the spread of disease from one animal to the next.

The size and species of the subjects must also be considered when administering mass injections. Different species and/or different sizes of animal within the same species typically require different dosages. Such animals may also require different needle lengths to be used, depending on their physical makeup.

A solution for providing multiple injections while providing a separate, sterile needle for each subject is described in U.S. patent application Ser. No. 10/868,764, filed Jun. 17, 2004 and assigned to the common assignee of the present invention. U.S. patent application Ser. No. 10/868,764, the disclosure of which is hereby incorporated in its entirety by reference, describes a fluid transfer device including a removable cartridge of needles that enables a user to quickly and efficiently perform multiple injections while providing a separate, sterile needle for each subject.

FIG. 1, to which reference is now made, is a simplified side view of such a device. A fluid transfer device, generally designated 100, comprises a pumping assembly 101 and a needle cartridge 102. Needle cartridge 102 is cylindrical in shape and comprises a multiplicity of needles 114, one of which, needle 114A, is shown extending from within cartridge 102. Pumping assembly 101 comprises a main housing 110, a main handle 106, a safety latch 108, and a piston assembly, generally designated 112. Piston assembly 112 comprises a piston pusher 214 and a fluid reservoir 204.

To operate fluid transfer device 100, the operator moves main handle 106 forward in order to rotate needle cartridge 102 and engage safety latch 108. Needle 114A is then extended by releasing safety latch 108, which also causes a predetermined amount of fluid to be prepared in fluid reservoir 204. At this point, the operator inserts needle 1 14A into a subject and then presses piston pusher 214 forward in order to force the fluid from reservoir 204, through needle 114A and into the subject.

### SUMMARY OF THE PRESENT INVENTION

An object of the present invention is to improve upon the prior art.

There is therefore provided, in accordance with a preferred embodiment of the present invention, a rapid injection device including a hand-held injection unit and an injection manager. The hand-held injection unit has a multiplicity of needles to inject at least one subject and the injection manager controls at least the injection unit according to injection parameters.

There is also provided, in accordance with a preferred embodiment of the present invention, a rapid injection device which includes an injection unit and an injection manager. The injection unit may be placed against the body of at least one subject to be injected and has a multiplicity of needles therein. The injection manager controls at least the extension and retraction of a different one of the needles into each subject.

Moreover, in accordance with a preferred embodiment of the present invention, the device also includes an external pump assembly to pump fluid to an extended one of the needles for an injection.

Still further in accordance with a preferred embodiment of the present invention, the injection parameters are at least dosage and needle extension.

Additionally, in accordance with a preferred embodiment of the present invention, the device also has modes of operation, wherein the modes of operation are at least automatic, semi-automatic, and manual.

Moreover, in accordance with a preferred embodiment of the present invention, the device also includes a removable cartridge to store the multiplicity of needles.

Further in accordance with a preferred embodiment of the present invention, the device also includes a belt mounted on a fixed revolving platform to store the multiplicity of needles.

Still further in accordance with a preferred embodiment of the present invention, the device also includes sealed containers, one for each of the multiplicity of needles, and means to check seals of the sealed containers.

Moreover, in accordance with a preferred embodiment of the present invention, the means include RFID tags embedded in the seals and RFID readers.

Additionally, in accordance with a preferred embodiment of the present invention, the device also includes a disposable pumping sub-system.

Moreover, in accordance with a preferred embodiment of the present invention, the disposable pumping sub-system includes components of the device that come in contact with said fluid.

Further, in accordance with a preferred embodiment of the present invention, the injection manager includes a needle extension controller to control the extension of a needle from a needle cartridge and a dosage controller to control the amount of fluid ejected from the needle.

There is also provided, in accordance with a preferred embodiment of the present invention, a method for performing rapid injections. The method includes receiving a mode of operation, receiving a dosage amount, receiving a needle extension length, extending a needle the needle extension length out a needle storage unit and pumping the dosage amount of fluid through the needle.

Further in accordance with a preferred embodiment of the present invention, the mode of operation is at least one of automatic, semi-automatic and manual.

Still further in accordance with a preferred embodiment of the present invention, the automatic mode of operation includes performing the extending and pumping in response to an activation signal, retracting the needle, and bringing a next needle into position.

Additionally in accordance with a preferred embodiment of the present invention, the semi-automatic mode of operation includes performing the extending in response to a first activation signal, performing the pumping in response to a second activation signal, retracting the needle and bringing a next needle into position.

Moreover, in accordance with a preferred embodiment of the present invention, the manual mode of operation includes performing the pumping in response to an activation signal, retracting the needle, bringing a next needle into position and performing the extending.

Further in accordance with a preferred embodiment of the present invention, the method also includes checking for an intact seal on a sealed needle container as a precondition for performing the extending.

Still further in accordance with a preferred embodiment of the present invention, the checking includes reading RFID tags embedded in the seals.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic drawing of a prior art rapid injection device;

FIG. 2 is a schematic drawing of a rapid injection device constructed and operative in accordance with a preferred embodiment of the present invention;

FIGS. 3, 4A, 4B, 4C, 5A and 5B are schematic illustrations of some of the entities depicted in FIG. 2;

FIG. 6 is a flow chart illustrating a process by which the device shown in FIG. 2 may operate;

FIG. 7 is a schematic illustration of an element to be attached to the entities shown in FIGS. 5A and 5B;

FIG. 8 is a flow chart illustrating a process by which the device shown in FIG. 2 may operate;

FIG. 9 is a schematic drawing of a rapid injection device constructed and operative in accordance with a preferred embodiment of the present invention; and

FIGS. 10A and 10B are schematic drawings of a grip assembly of a rapid injection device and revolving needle platform constructed and operative in accordance with a preferred embodiment of the present invention.

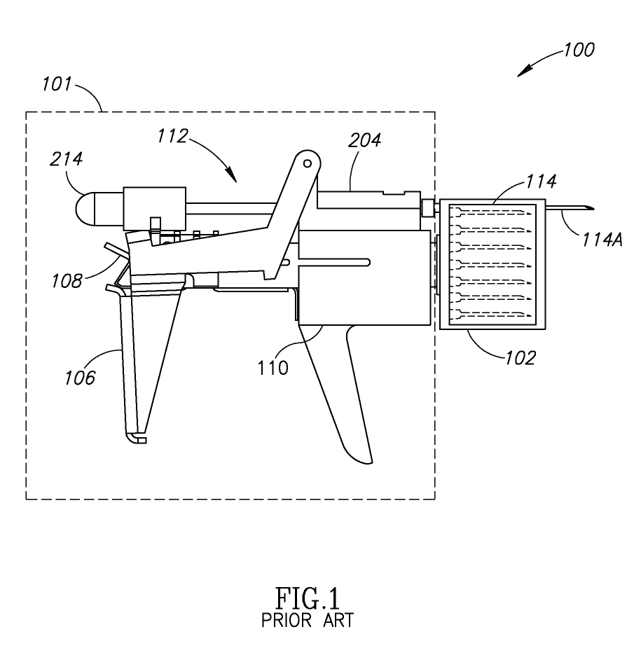

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

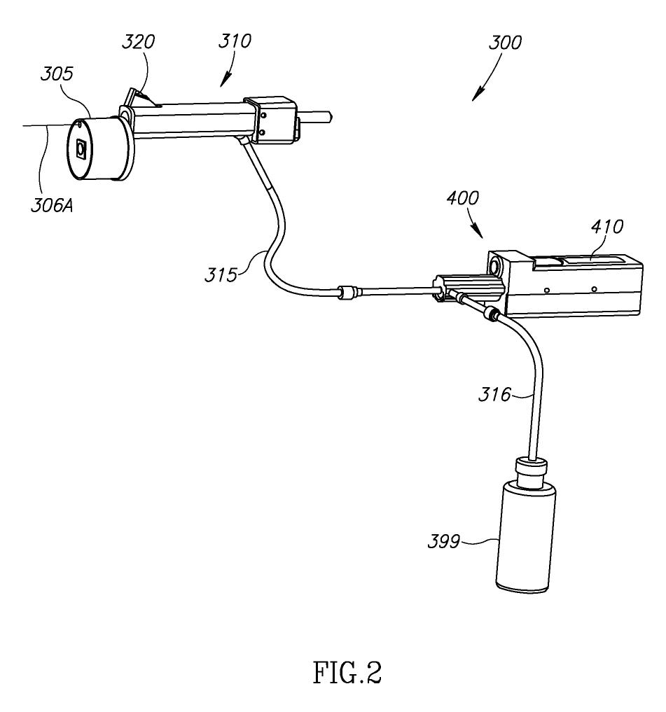

Reference is now made to FIG. 2, which shows a novel rapid injection device 300 constructed and operative in accordance with a preferred embodiment of the present invention. As shown in FIG. 2, rapid injection device 300 may comprise a grip assembly 310, a pumping assembly 400 and a fluid tank 399. Connecting tube 315 may connect grip assembly 310 to pumping assembly 400, and fluid tube 316 may connect fluid tank 399 to pumping assembly 400.

In accordance with a preferred embodiment of the present invention, pumping assembly 400 may comprise a micro controller (not shown) and a control panel 410 that may enable an operator to set various settings and select a mode of operation. Grip assembly 310 may comprise a rocker switch 320 and a replaceable needle cartridge 305. Needle cartridge 305 may be a cylindrical cartridge with a multiplicity of needles 306, where 306A is shown extended. In order to perform injections, an operator may first dial a desired dosage and/or a needle extension length and/or mode of operation through control panel 410. This may cause pumping assembly 400 to pump the desired dosage from tank 399 to grip assembly 310. When the operator may place needle cartridge 305 against a subject's body and may press rocker switch 320, grip assembly 310 may extend needle 306A to the designated length and may inject fluid into the subject at the desired depth under the subject's skin. The further needle 306A is extended, the deeper the injection may occur.

It will be appreciated that pumping assembly 400 and fluid tank 399 may stay in one general location, for example, attached to a belt assembly, as the user may move from one subject to another with grip assembly 310. Grip assembly 310 may be relatively light and rocker switch 320 may be relatively easy to push, making device 300 relatively easy to operate with one hand. This may enable the operator to use the other hand as necessary, such as to touch or control the subject. This may be important, especially when injecting large animals or human beings.

It will be appreciated that, by dialing in the desired dosage and/or needle extension length, device 300 may enable an operator to relatively perform mass injections.

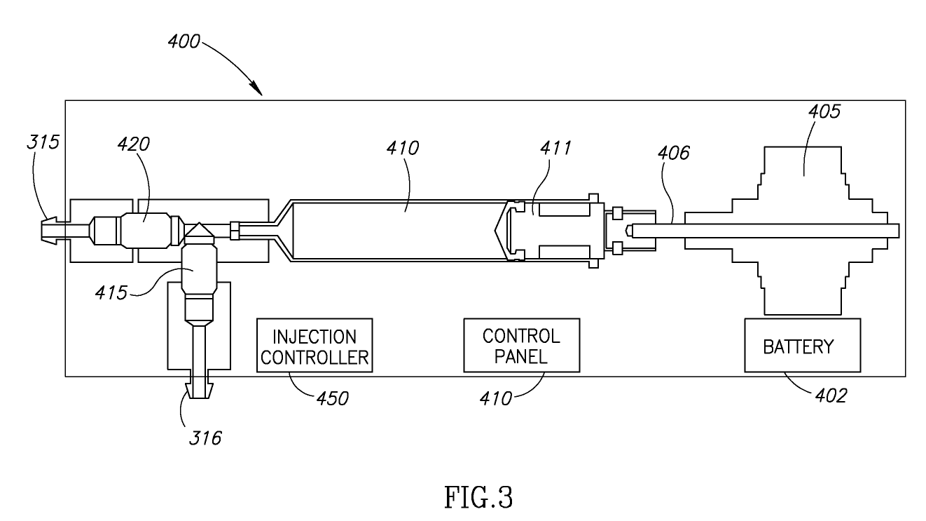

Reference is now made to FIG. 3 which illustrates a simplified top view of pumping assembly 400. Pumping assembly 400 may comprise a battery 402, a DC motor 405, a syringe 410, an inlet valve 415 and an outlet valve 420. A motor shaft 406 may extend from motor 405 and may connect with a piston 411, located within syringe 410. Inlet valve 415 may connect with fluid tube 316, and outlet valve 420 may connect with connecting tube 315. In accordance with a preferred embodiment of the present invention, pumping assembly may also comprise control panel 410 and an injection manager 450.

In accordance with a preferred embodiment of the present invention, both inlet valve 415 and outlet valve 420 may be one way valves. Inlet valve 415 may be configured to only allow fluid to flow through fluid tube 316 from fluid tank 399 (FIG. 2). Outlet valve 420 may be configured to only allow fluid to flow outward through connecting tube 315 to grip assembly 310.

DC motor 405 may extend and retract DC motor shaft 406, thus moving piston 411 backwards and forwards within syringe 410. Piston 411 may be coated with a flexible material and may be tightly fitted within syringe 410. Accordingly, moving piston 411 backwards may cause a vacuum within syringe 410 and may thus draw fluid in through inlet valve 415 from fluid tank 399 (not shown). Moving piston 411 forwards may force fluid through outlet valve 420 towards grip assembly 31 0.

In accordance with a preferred embodiment of the present invention, rapid injection device 300 (FIG. 2) may be configured such that pumping assembly 400 and fluid tank 399 may be attached to a belt or harness assembly to be worn by an operator while performing injections. For this embodiment, battery 402 may provide the electrical power. In accordance with an alternative embodiment of the present invention, a connection to an external power source may be provided to replace or charge battery 402.

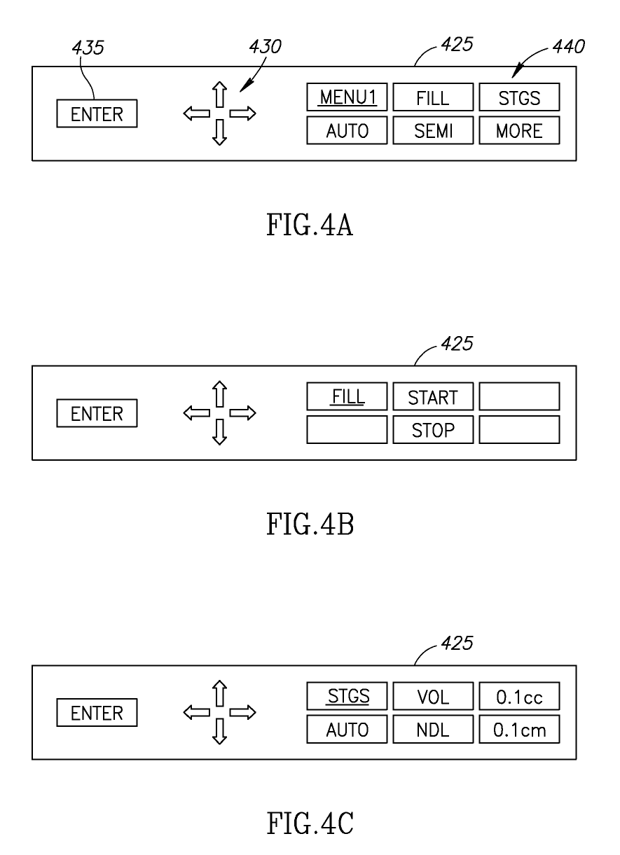

In accordance with a preferred embodiment of the present invention, injection controller 450 may control the various modes of operation for rapid injection device 300. Control panel 410 may be used by the operator to input control settings for injection controller 450. Reference is now made to FIGS. 4A-4C which illustrate how control panel 410 may be operated.

As shown in FIG. 4A, control panel 410 may comprise navigation arrows 430, an enter key 435 and a display 440. The four navigation keys 430 may be labeled with symbols indicating up, down, right and left. It will be appreciated that display 440 may be an LCD display or any other suitable technology. In accordance with an alternative embodiment of the present invention, display 440 may also be a touch screen.

In accordance with an exemplary embodiment of the present invention, display 440 may display a menu and its available options, where the name of the menu may be displayed in the upper left corner of display 440, and its associated options may be displayed in the remaining area of display 440.

For example, FIG. 4A may show an initial menu to be displayed when accessing control panel 410. The name of the menu may be “MENU1”, and its associated options may be “FILL”, “STGS”, “AUTO”, “SEMI” and “MORE”. MENU1 may be navigated by pressing navigation keys 430. Focus may move from option to option in accordance with the arrow direction of the navigation key 430 as pressed by the operator. Menus and menu option may be selected by pressing enter key 435. For example, pressing enter 435 while focus is on “FILL” may access a new sub-menu named “FILL”.

FIG. 4B shows control panel 410 with an exemplary “FILL” menu. The “FILL” menu may be used to fill rapid injection device 300 with fluid prior to use. It may also be used to rinse out rapid injection device 300 with a cleaning fluid after use. Two options may be displayed: “START” and “STOP”. Navigation keys 430 may be pressed to move focus to the option labeled “START”. Subsequently pressing enter key 435 may then prompt injection controller to instruct DC motor 405 (FIG. 3) to begin moving piston 411 (FIG. 3) backwards and forwards to continuously pump fluid, such as cleaning fluid, from fluid tank 399 (FIG. 2) to grip assembly 310 (FIG. 2) as described hereinabove. Navigating to “STOP” and pressing enter key 435 may have the opposite effect and the pumping action may cease.

It will be appreciated that selecting the “FILL” option when fluid tank 399 is disconnected will pump air through rapid injection device 300. Accordingly, in accordance with an exemplary embodiment of the present invention such “air pumping” may be performed after, or instead of, rinsing of cleaning fluid after use.

Pressing enter key 435 while focus is on a sub-menu name may return display 440 to the previous menu. For example, when the “FILL” menu is displayed, pressing enter key 435 will return display 440 to the “MENU1” menu.

FIG. 4C shows display 410 with the “STGS” sub-menu displayed. This sub-menu may include two settings to be set prior to injecting a subject with fluid. The “VOL” setting refers to the volume to be pumped for each injection. A minimum value may be displayed by default, for example, “0.1 cc”. To adjust this value, the operator may use navigation keys 430 to navigate to the “VOL” option and then press enter key 435. The operator may then use the up and down navigation keys 430 to increase or decrease the volume to be injected. In accordance with an exemplary embodiment of the present invention, the volume may be increased by increments of 0. I cc. It will be appreciated that other increments may be defined as well.

Injection controller 450 may use this volume setting to determine the positioning of piston 411 (FIG. 3) when it may move backward to fill syringe 410 with fluid prior to an injection. It will be appreciated that, as described hereinbelow, the volume of fluid injected into a subject may be equal to the volume of fluid in syringe 410 prior to the associated pumping action.

A needle extension length for injections may be set in a similar fashion by selecting “NDL” from the “STGS” sub-menu. In accordance with an exemplary embodiment of the present invention, the needle extension length may be increased by increments of 0.1 cm. It will be appreciated that other increments may be defined as well. The implementation of the needle extension length setting will be discussed in greater detail hereinbelow.

The “MORE” option from menu “MENU1” may display usage statistics. For example, display 440 may show the number of injections performed and/or the total volume of fluid injected.

Rapid injection device 300 may have three modes for injections: automatic, semi-automatic and manual, selected via the “AUTO”, “SEMI” and “MAN” options from the “MENU1” menu. Automatic mode may entail a one step operation in which the operator may press rocker switch 320 once in order to extend a new needle, insert it into the subject, and inject the required fluid. Semi-automatic mode may entail a three step operation: the operator may press rocker switch 320 to extend a needle, the operator may then manually insert the needle into a subject, and then the operator may press rocker switch 320 to inject the fluid, after which the needle may be retracted. Manual mode may be similar to semi-automatic mode, except that a new needle for use with the next subject may be extended immediately after the fluid is injected.

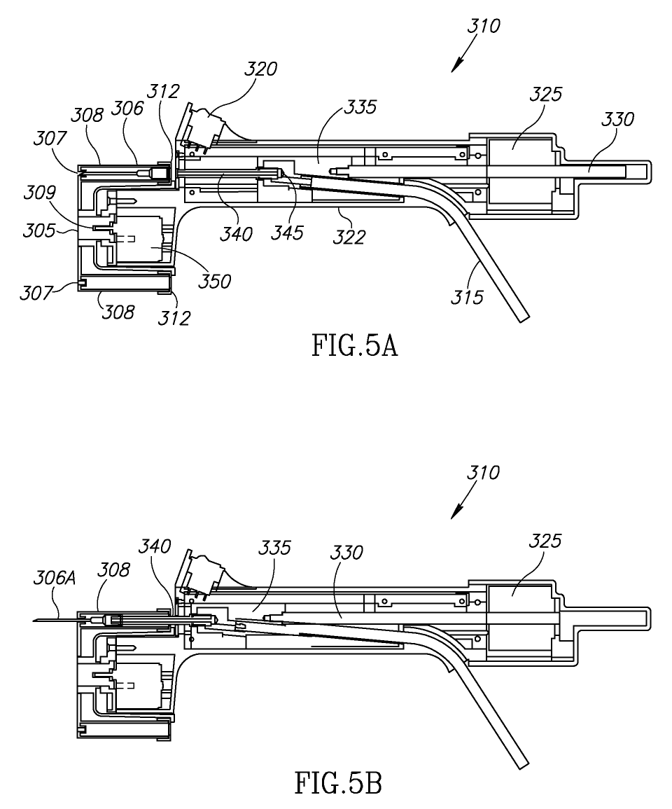

Reference is now made to FIG. 5A which shows a simplified side view of grip assembly 310. Grip assembly 310 may comprise a housing 322, needle cartridge 305, rocker switch 320, a DC motor 325, a lead screw 330, a movable housing 335 and a fluid chamber 340. Connecting tube 315 may be attached to fluid chamber 340 via a connecting seal 345.It will be appreciated that any suitable motor for turning lead screw 330 may be used in place of DC motor 325.

It will be appreciated that the general shape of housing 322 may be suitable for grasping in one hand. The operator's thumb may rest on or near rocker switch 320 and the other four fingers may comfortably grip the underside of housing 322. Rocker switch 320 may be configured in such a manner such that it may be pressed either forward or backward.

In accordance with an exemplary embodiment of the present invention, pressing rocker switch 320 forward may be referred to hereinbelow as pressing “A”, and similarly, pressing rocker switch 320 backward may be referred to hereinbelow as pressing “B”. It will be appreciated that rocker switch 320 is exemplary; any suitable electronic device or method for transmitting two distinct requests may be used.

It will also be appreciated that connecting tube 315 may comprise not only a channel suitable for the transfer of fluids, but it may also comprise one or more electric wires that may transfer electric current and electronic instructions between the micro-controller in pumping assembly 400 and grip assembly 310.

Needle cartridge 305 may comprise a multiplicity of needle housings 308 each storing one needle 306, and a stepper motor 350. Stepper motor 350 may be capable of rotating needle cartridge 305 upon its axis 309 in order to position a needle 306 opposite fluid chamber 340. Exterior needle seals 307 and interior needle seals 312 may cover exit and entry apertures respectably in each needle housing 308. Needle seals 307 and 312 may be of any suitable material, for example paper or plastic, which may typically be used to seal sterile medical instruments. As will be described hereinbelow, RFID tags may also be embedded in needle seals 312.

It will be appreciated that needle cartridge 305 and its contents may be prepared in a sterile environment and that needle seals 307 and 312 may protect needles 306 from contamination. In accordance with an exemplary embodiment of the present invention, needle cartridge 305 may comprise 24 needles 306. It will, however, be appreciated that other amounts of needles 306 may be stored as well.

FIG. 5B illustrates grip assembly 310 with one needle, here labeled 306A, extended. Motor 325 may have moved lead screw 330 forward, pushing movable housing 335 and fluid chamber 340 forward as well. This may push needle 306A from within its needle housing 308, breaking needle seals 307 and 312 (not shown) in the process. In addition, fluid chamber 340 may make contact with needle 306A, enabling fluid to pass into needle 306A. It will be appreciated that the extent to which needle 306 may extend from within needle housing 308 may be determined by the amount that lead screw 330 has moved forward. In such manner, injection manager 450 (FIG. 3) may implement an operator's selected setting for needle extension length. It will be appreciated that injection manager 450 may also retract needle 306 by moving lead screw 330 in the opposite direction.

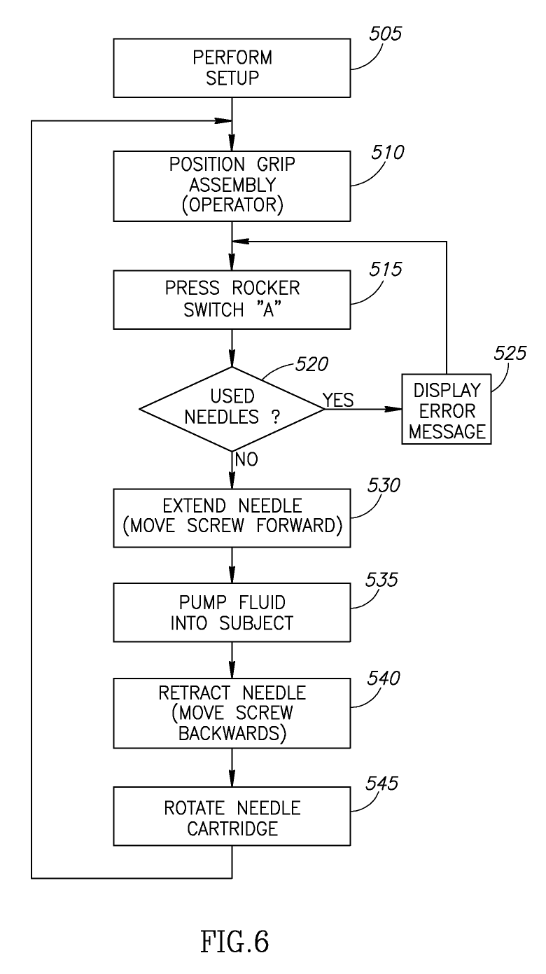

As described hereinabove, rapid injection device 300 may have three modes of operation for injections: automatic, semi-automatic and manual. FIG. 6, to which reference is now made, illustrates the steps executed during the automatic mode of operation.

Setup procedures may be performed (step 505) as described hereinabove. For example, the volume of fluid may be set to 0.2 cc and the length of the needle may be set to 0.3 cm. The operator may also have selected the “FILL” operation as described hereinabove and to fill connecting tube 315 and fluid chamber 340 with fluid. The automatic mode of operation may also be selected.

Grip assembly 310 may be positioned (step 510) by the operator such that the top portion of needle cartridge 305 may touch the subject at a site suitable for injection. Operator may press “A” (step 515) on rocker switch 320 to initiate an injection.

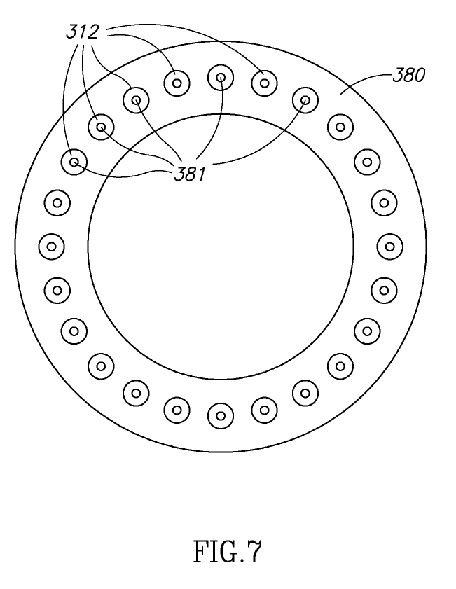

Injection manager 450 may check (step 520) needle seal 312 in order to determine whether or not needle 306 may have been previously used or otherwise contaminated. In a preferred embodiment of the present invention, such checking may be performed using RFID technology. FIG. 7, to which reference is now briefly made, shows an exemplary needle cartridge sealing unit 380 which may be fitted over the interior portion of needle cartridge 305 to position interior needle seals 312 over the entry apertures for each needle housing 308. In each interior needle seal 312, an RFID tag 381 may be embedded.

An RFID reader unit may be mounted on or near fluid chamber 340 in a manner suitable for reading the associated RFID tag 381 of each interior needle seal 312 when its associated needle housing 308 may be positioned opposite fluid chamber 340. It will be appreciated that if an RFID tag 381 may be read, then it may be reasonable to assume that the associated needle seal 312 may still be intact and needle 306 may be assumed to still be sterile and available for use. If RFID tag 381 may not be read, an error message may be displayed (step 525) on display 440 and needle cartridge 305 may be advanced to position the next needle housing 308 opposite fluid chamber 340. The operator may have to press (step 515) “A” again or replace needle cartridge 305 before continuing.

Assuming that needle 306 may not have been used previously or otherwise contaminated, device 310 may extend (step 510) needle 306 into the subject. Linear motor 325 may move screw 330 forward, which, in turn may slide movable housing 335 as well. Fluid chamber 340 may thus be pushed into needle cartridge 305, making contact with needle 306 and pushing it out through needle seal 307 and into the subject. It will be appreciated that, as disclosed in previously discussed U.S. patent application Ser. No. 10/868,764, needle 306 maybe outfitted with a flexible nipple enabling fluid chamber 340 to fit snugly and prevent leakage.

Pumping assembly 400 may pump (step 535) a single dosage of fluid, as described hereinabove, into connecting tube 315. This may cause a single dosage of fluid to move from fluid chamber 340 into needle 306 and from there into the subject.

Needle 306 may then be retracted (step 540) from the subject. Linear motor 325 may move screw 330 backward, which, in turn, may slide movable housing 335 and fluid chamber 340 back as well. As disclosed in U.S. patent application Ser. No. 10/868,764, needle housing 308 may be outfitted with a spring mechanism to retract needle 306 once fluid chamber 340 has exited from with housing 308.

Needle cartridge 305 may then be rotated (step 545) by stepper motor 350 (FIG. 5A) in order to align a different needle 306 opposite fluid chamber 340. Operation may then continue again from step 510 with a different subject.

It will be appreciated that steps 530-545 may be performed in rapid succession with no need of intervention by the operator.

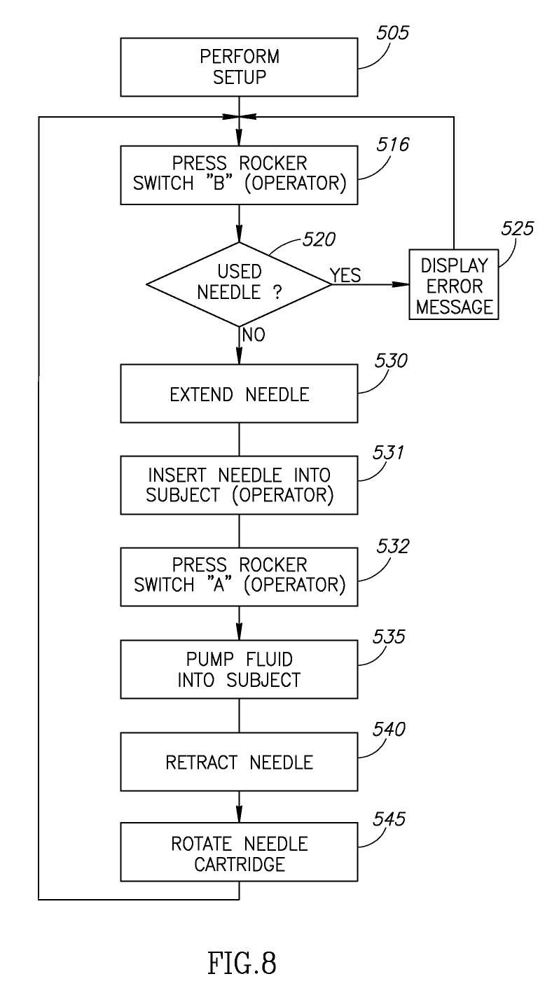

FIG. 8, to which reference is now also made, illustrates the steps executed during the semi-automatic mode of operation. As with the automatic mode of operation, setup procedures may be performed (step 505), one of which is the selection of the semi-automatic mode of operation.

The operator may press (step 516) “B” on rocker switch 320. As with the automatic mode of operation, interior needle seal 312 may be checked (step 520) for breakage, and an error message displayed (step 525) in the event that a broken interior needle seal 312 may be detected. If needle seal 312 may have been broken, needle cartridge 305 may advance in order to place the next needle 306 in position for an injection. The operator may press (step 515) “B” again or replace needle cartridge 305 before continuing.

As with the automatic mode of operation, assuming that needle 306 may not be contaminated, needle 306 may be extended (step 530). However, such extension may not necessarily be into the subject's body, and pumping assembly 400 may not automatically pump fluid to grip assembly 310. Instead, the operator may manually insert (step 531) extended needle 306 into the subject.

The operator may then press (step 532) “A” on rocker switch 320 to execute steps 535-545 as described hereinabove during the discussion of the automatic mode of operation.

It will be appreciated that the semi-automatic mode of operation may afford a higher degree of accuracy regarding the location of the insertion site for needle 306.

In accordance with an alternative, preferred embodiment of the present invention, a pumping sub-system that comes in contact with the fluid may be disposable. Instead of cleaning this sub-system by pumping cleaning fluid through it, the removable sub-system may be removed and replaced after use. This pumping sub-system may comprise components from both pumping assembly 400 and grip assembly 310.

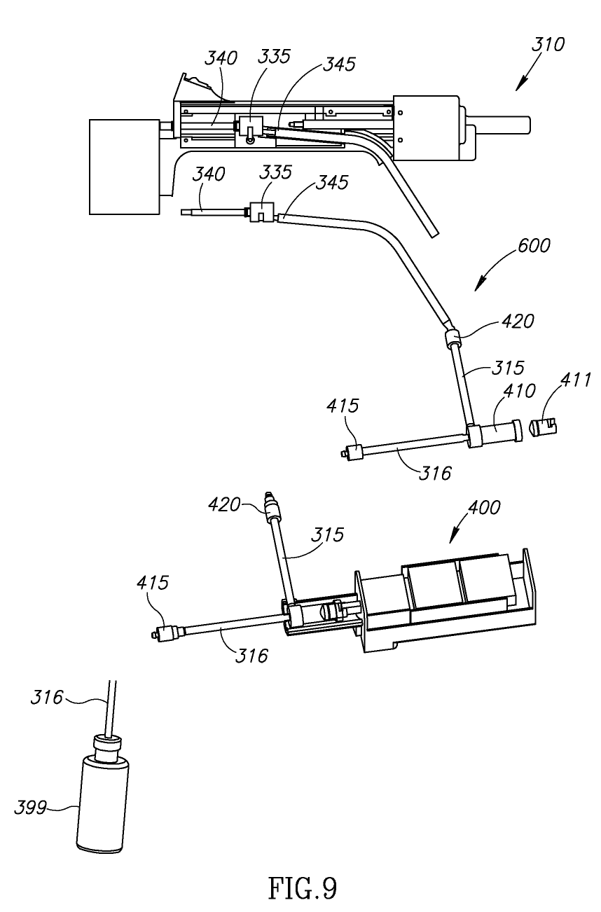

FIG. 9 to which reference is now made illustrates the various components comprising pumping sub-system 600. Pumping sub-system 600 may comprise the following parts from pumping assembly 400: syringe 410, piston 411, valves 415 and 420, fluid tube 316 and connecting tube 315. Pumping sub-system 600 may also comprise the following parts from grip assembly 310: movable housing 335, connecting seal 345 and fluid chamber 340.

It will be appreciated that a disposable pumping sub-system 600 may save operation time and lessen the possibility of contamination within rapid injection device 300.

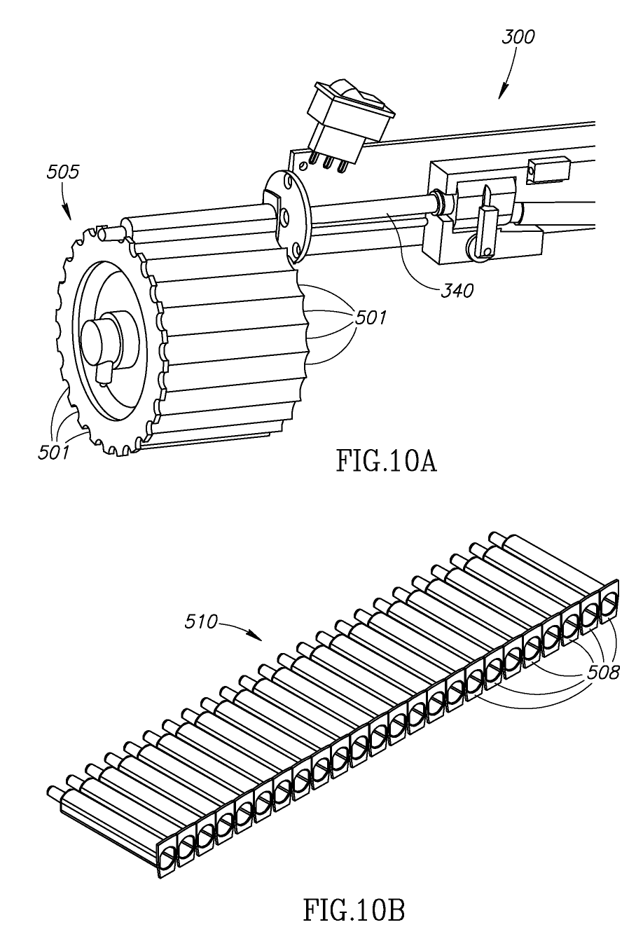

In an alternate preferred embodiment of the present invention, removable needle cartridge 305 may be replaced by a fixed revolving platform for mounting needles. FIGS. 10A and 10B, to which reference is now made, illustrate how such a fixed revolving needle platform 505 may be implemented as part of rapid injection device 300.

Fixed revolving needle platform 505 (FIG. 10A) may be permanently attached to rapid injection device 300 and may comprise a multiplicity of mounting grooves 501. Disposable needle belt 510 (FIG. 10B) may comprise a corresponding multiplicity of needle assemblies 508, one for each mounting groove 501. Disposable needle belt 510 may be mounted on fixed revolving needle platform 505 by placing each needle assembly 508 in a corresponding mounting groove 501. It will be appreciated that any suitable closing mechanism may be used to lock disposable needle belt 508 in place on fixed revolving needle platform 505. In an exemplary embodiment of the present invention each mounting groove 501 may comprise extending arcs that may firmly grip needle assemblies 508.

Fixed revolving needle platform 505 with attached needle belt 510 may be generally operated in the same manner as needle cartridge 305. Fixed revolving needle platform 505 may be rotated in order to line up a needle assembly 508 opposite fluid chamber 340. Needle assembly 508 may generally comprise the same components as needle housing 308 and may generally operate in the same manner.

It will be appreciated that using fixed revolving needle platform 505 may provide a more efficient use of time and materials than needle cartridge 305. For example, fixed revolving needle platform may require less manufacturing investment per needle used. Furthermore, it may be more convenient for the operator to carry spare disposable needle belts 510 instead of a like number of needle cartridges 305.

While certain features of the invention have been illustrated and described herein, many modifications, substitutions, changes, and equivalents will now occur to those of ordinary skill in the art. It is, therefore, to be understood that the appended claims are intended to cover aft such modifications and changes as fall within the true spirit of the invention.

What is claimed is:

1. A method for performing injections, the method comprising:
receiving a selection of a mode of operation;
receiving a selection of a dosage amount;
receiving a selection of a needle extension length;
extending a needle said needle extension length out a needle storage unit; and
pumping said dosage amount of fluid through said needle.

2. The method according to claim 1 and wherein said mode of operation is at least one of automatic, semi-automatic and manual.

3. The method according to claim 2 and wherein said automatic mode of operation comprises:
performing said extending and pumping in response to an activation signal;
retracting said needle; and
bringing a next needle into a predefined injection position.

4. The method according to claim 2 and wherein said semi-automatic mode of operation comprises:
performing said extending in response to a first activation signal;
performing said pumping in response to a second activation signal;
retracting said needle; and
bringing a next needle into a predefined injection position.

5. The method according to claim 2 and wherein said manual mode of operation comprises:
performing said pumping in response to an activation signal;
retracting said needle;
bringing a next needle into a predefined injection position; and
performing said extending.

6. The method according to claim 1 and also comprising checking for an intact seal on a sealed needle container as a precondition for performing said extending.

7. The method according to claim 6 and wherein said checking comprises reading RFID tags embedded in said seals.

8. A rapid injection device comprising:
a hand-held injection unit having a multiplicity of needles to inject at least one subject; and
an injection manager to control at least said injection unit, wherein said injection manager is configured to
receive a selection of a mode of operation;
receive a selection of a dosage amount;
receive a selection of a needle extension length;
cause said injection unit to extend a needle said needle extension length out a needle storage unit; and
cause said injection unit to pump said dosage amount of fluid through said needle.

9. The device according to claim 8 and also comprising:
an external pump assembly to pump fluid to an extended one of said needles for an injection.

10. The device according to claim 8 and also having modes of operation wherein said modes of operation are at least automatic, semi-automatic, and manual.

11. The device according to claim 8 and also comprising a removable cartridge to store said multiplicity of needles.

12. The device according to claim 8 and also comprising a belt mounted on a fixed revolving platform to store said multiplicity of needles.

13. The device according to claim 8 and also comprising sealed containers, one for each of said multiplicity of needles, and means to check seals of said sealed containers.

14. The device according to claim 13 and wherein said means comprise RFID tags embedded in said seals and RFID readers.

15. The device according to claim 9 and also comprising a disposable pumping sub-system.

16. The device according to claim 15 and wherein said disposable pumping sub-system comprises components of said device that come into contact with said fluid.

17. The device according to claim 8 and wherein said injection manager comprises:
a needle extension controller to control the extension of a needle from a needle cartridge; and
a dosage controller to control the amount of fluid ejected from said needle.
